(19) United States
(12) Patent Application Publication
(10) Pub. No.: US 2004/0116529 A1
(43) Pub. Date: Jun. 17, 2004

(54) Anticancer and chemopreventative agents and methods of use thereof

(76) Inventors: Wayne B. Harris, Stone Mountain, GA (US); Kimberly M. Jackson, Stone Mountain, GA (US)

Correspondence Address: Christopher B Linder, Thomas Kayden Horstemeyer & Risley, Suite 1750, 100 Galleria Parkway NW, Atlanta, GA, 30339-5948, US

(21) Appl. No.: 10/472,662

(22) PCT Filed: Mar. 29, 2002
(86) PCT No.: PCT/US02/09978

(30) Foreign Application Priority Data
Mar. 29, 2001 (US) 60279865

(51) Int. Cl. A61K 31/192

(52) U.S. Cl. 514/569

(57) ABSTRACT

Pharmaceutical compositions and methods of treating conditions such as cancer and cancer-related conditions are disclosed. A representative method includes administering to a host in need of treatment an effective amount of at least one effector agent. Another method includes prophylactically treating cancer or cancer-related conditions by administering to a host in need of treatment an effective amount of at least one effector agent. Still another method includes methods of down regulating androgen receptors and/or prostate-specific antigen (PSA) levels in human prostate cancer cells by administering an effective amount of at least one effector agent to a host in need of treatment.

### CROSS-REFERENCE TO RELATED APPLICATION

This application claims priority to copending U.S. provisional application entitled, “Suppressors Of Androgen Receptor Protein Expression As Chemopreventative Antineoplastic And Antiprogressive Agents In Prostate Cancer,” having ser. No. 60/279,865, filed Mar. 29, 2001, which is entirely incorporated herein by reference.

### TECHNICAL FIELD

The present invention is generally related to compositions and agents and methods for administration to hosts and, more particularly, is related to a compositions and agents designed for treatment of cancer and cancer related conditions and methods of administration thereof.

### BACKGROUND

Cancer can be defined as an abnormal growth of tissue characterized by a loss of cellular differentiation. This term encompasses a large group of diseases in which there is an invasive spread of undifferentiated cells from a primary site to other parts of the body where further undifferentiated cellular replication occurs, which eventually interferes with the normal functioning of tissues and organs.

Cancer can be defined by four characteristics which differentiate neoplastic cells from normal ones: (1) clonality—cancer starts from genetic changes in a single cell which multiplies to form a clone of neoplastic cells; (2) autonomy—biochemical and physical factors that normally regulate cell growth, do not do so in the case of neoplastic cells; (3) anaplasia—neoplastic cells lack normal differentiation which occurs in nonmalignant cells of that tissue type; (4) metastasis—neoplastic cells grow in an unregulated fashion and spread to other parts of the body.

Each cancer is characterized by the site, nature, and clinical cause of undifferentiated cellular proliferation. The underlying mechanism for the initiation of cancer is not completely understood; however, about 80% of cancers may be triggered by external stimuli such as exposure to certain chemicals, tobacco smoke, ultra violet rays, ionizing radiation, and viruses. Development of cancer in immunosuppressed individuals indicates that the immune system is an important factor controlling the replication and spread of cancerous cells throughout the body.

The high incidence of cancer in certain families, though, suggests a genetic disposition towards development of cancer. The molecular mechanisms involved in such genetic dispositions fall into a number of classes including those that involve oncogenes and suppressor genes.

Proto-oncogenes are genes that code for growth promoting factors necessary for normal cellular replication. Due to mutation, such proto-oncogenes are inappropriately expressed—and are then termed oncogenes. Oncogenes can be involved in malignant transformation of the cell by stimulating uncontrolled multiplication.

Suppressor genes normally act by controlling cellular proliferation through a number of mechanisms including binding transcription factors important to this process. Mutations or deletions in such genes contribute to malignant transformation of a cell.

Malignant transformation develops and cancer results because cells of a single lineage accumulate defects in certain genes such as proto-oncogenes and suppressor genes responsible for regulating cellular proliferation. A number of such specific mutations and/or deletions must occur in a given cell for initiation of uncontrolled replication. It is believed that genetic predisposition to a certain type of cancer results from inheritance of genes that already have a number of mutations in such key regulatory genes and subsequent exposure to environmental carcinogens causes enough additional key mutations or deletions in these genes in a given cell to result in malignant transformation. Changes in other types of genes could further the ability of tumors to grow, invade local tissue, and establish metastases at distant body sites.

Current treatments of cancer and related diseases have limited effectiveness and numerous serious unintended side effects. Cancer therapy is currently divided into many categories: surgery, radiation therapy, bone marrow therapy, stem cell transplantation, chemotherapy, gene therapy, hormonal therapy, immunotherapy, and antiangiogenic therapy. These treatments have progressed only incrementally during more than thirty years of intensive research to discover the origins of cancer and devise improved therapies for cancer and related diseases.

Current research strategies emphasize the search for effective therapeutic modes with less risk, including the use of natural products and biological agents. This change in emphasis has been stimulated by the fact that many of the consequences, to both patients and their offspring, of conventional cancer treatment result from their actions on genetic material and mechanisms. Efforts continue to discover both the origins of cancer at the genetic level and correspondingly new treatments, but such interventions also may have serious unanticipated effects.

With the exception of skin cancer, prostate cancer is the most common cause of cancer in men. The risk of developing prostate cancer increases as men get older. In general, the chance of a man developing invasive prostate cancer during his lifetime is 1 in 6 or 15.4%. At the age of 50, a man has a 42% chance of developing prostate cancer and 2.9% of dying from the disease. Physicians usually detect cancers by finding a lump in the prostate gland, which is a walnut shaped structure that helps to maintain the viability of sperm. Such lumps may be discovered during a routine checkup or an examination prompted by a patient's complaint of sudden urinary discomfort, or occasional impotence. While advances in early diagnosis and treatment of locally confined tumors have been achieved, prostate cancer is incurable once it has metastasized. Patients with metastatic prostate cancer on hormonal therapy will eventually develop an androgen-refractory (androgen independent) state that will lead to disease progression and death.

Thus, a heretofore unaddressed need exists in the industry to address at least the aforementioned deficiencies and/or inadequacies in regard to preventing and treating cancer and related diseases, and particularly prostate cancer.

### SUMMARY OF THE INVENTION

Briefly described, embodiments of the present invention include representative methods to treat conditions such as cancer and cancer-related conditions. A representative method includes administering to a host in need of treatment an effective amount of at least one effector agent. Another method includes prophylactically treating one or more of the conditions listed above by administering to a host in need of treatment an effective amount of at least one effector agent. Still other methods include down regulating androgen receptors and/or prostate-specific antigen (PSA) levels in human prostate cancer cells by administering an effective amount of at least one effector agent to a host in need of treatment.

Alternate embodiments of the present invention also involve pharmaceutical compositions having at least one effector agent in combination with a pharmaceutically acceptable carrier. The effector agent is present in a dosage level effective to treat conditions such as cancer and cancer-related conditions.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a diagram that illustrates a dibenzoylmethane-type structure as structure A1 and dibenzoylmethane as structure A2 of the present invention.

FIG. 2 is a diagram that illustrates representative functional groups Ar₁ and Ar₂ of the dibenzoylmethane-type structure illustrated in FIG. 1.

### DETAILED DESCRIPTION

The present invention provides compositions and agents that can be used to treat hosts having cancer and cancer-related conditions. In addition, the present invention provides chemopreventative compositions and chemopreventative methods of treating hosts that are predisposed to cancer and cancer-related conditions.

In particular, embodiments of the present invention include methods of down regulating androgen receptors and/or prostate-specific antigen (PSA) levels in human prostate cancer cells by administering an effective amount of at least one effector agent to a host in need of treatment. Additional embodiments of the present invention include pharmaceutical compositions including one or more effector agents in combination with a pharmaceutically acceptable carrier. The effector agents are present in a dosage level effective to down regulate androgen receptors present in host cells. Other embodiments of the present invention include methods of treating cancer by administering to a host in need of treatment an effective amount of at least one effector agent. Still other embodiments of the present invention include chemopreventative methods of prophylactically treating cancer and cancer-related conditions by administering to a host in need of treatment an effective amount of at least one effector agent.

As used herein, the term “host” includes both humans, mammals (e.g., cats, dogs, horses, etc.), and other living species that are in need of cancer and/or cancer related treatments. Hosts that are “predisposed” to cancer and cancer-related conditions can be defined as hosts that do not exhibit overt symptoms of one or more of these conditions but that are genetically, physiologically, or otherwise at risk of developing one or more of these conditions. Thus, compositions and effector agents of the present invention can be used prophylactically as chemopreventative agents for these conditions. Further, a “composition” or “agent” can include one or more chemical compounds and/or agents, as described below.

As used herein, “host cells” include non-cancerous and cancerous cells. “Cancerous cells” include, but are not limited to, cancer cells, neoplastic cells, neoplasia, tumors, and tumor cells, which exhibit relatively autonomous growth, so that they exhibit an aberrant growth phenotype, characterized by a significant loss of control of cell proliferation.

As used herein, “treat”, “treating”, and “treatment” are an approach for obtaining beneficial or desired clinical results. For purposes of embodiments of this invention, beneficial or desired clinical results include, but are not limited to, alleviation of symptoms, diminishment of extent of disease, stabilization (i.e., not worsening) of disease, preventing spread (i.e., metastasis) of disease, delaying or slowing of disease progression, amelioration or palliation of the disease state, and remission (partial or total) whether detectable or undetectable. In addition, “treat”, “treating”, and “treatment” can also mean prolonging survival as compared to expected survival if not receiving treatment.

As used herein, “androgen receptor” refers to a protein whose function is to specifically bind to androgen and, as a consequence of the specific binding, recognizes and binds to an androgen response element (ARE). Thereafter, the androgen receptor is capable of regulating transcriptional activity. In non-cancerous cells the androgen receptor is activated by androgen, but in non-normal cells (including cancerous cells) the androgen receptor may be activated by non-androgenic agents, including hormones (e.g., substances that have specific regulatory action) other than androgens. In general, activation of androgen receptors through non-androgenic agents or ligands such as an increase in endogenous coactivators or a decrease in endogenous corepressors may contribute to the progression of prostate cancer in the hormone independent state.

Encompassed in the term “androgen receptor” are wild and mutant forms of an androgen receptor. Mutant forms of the androgen receptor are considered within the scope of embodiments of this invention as long as the function of mutant androgen receptor is sufficiently preserved. In addition, mutant androgen receptors include androgen receptors with amino acid additions, insertions, truncations and deletions, as long as the function is sufficiently preserved.

“Down regulation” or “down regulating” can be defined as a decrease in the number of ligand receptors or other cellular proteins within or on the surface of a host cell. Down regulation occurs after host cells have been exposed to an effector agent, either as a result of a direct interaction of the effector agent with the receptor or other protein, or through indirect interactions.

Down regulation of cellular proteins may be induced by any cellular perturbation that results in a decrease in protein production or synthesis or an increase in protein degradation. Cellular protein synthesis occurs through the sequential steps of transcription and translation. Transcription is defined as the synthesis of ribonucleic acid (RNA) from a deoxyribonucleic acid (DNA) template. Translation is defined as the synthesis of a protein directed by messenger RNA (mRNA). In general, lysosomes and ubiquitin are the two major pathways for the degradation of cellular proteins. Proteins such as the androgen receptor, that undergo ubiquitination, are degraded by a subcellular protein complex known as the proteosome.

The number of androgen receptors within a cell is determined by a dynamic equilibrium between synthesis and degradation. Therapeutic down regulation of the androgen receptor can occur by exposure to effector agents that disrupt protein synthesis at the DNA or RNA level. Additionally, therapeutic down regulation of the androgen receptor can also occur by exposure to effector agents that enhanced protein degradation, either via the proteosomal pathway or methods of protein degradation.

An “effector agent” can be defined as any small molecule that interacts with a receptor, either directly or indirectly, in a manner that alters its ability to bind a ligand. A positive effector enhances binding activity while a negative effector reduces it.

In particular, effector agents shown to modulate androgen receptor levels can include, but are not limited to, beta-diketones, cadmium, 1(alpha), 25-dihyroxyvitamin D₃, fenretinide, flufenamic acid, Chinese herbal mixture PC-SPES, quercetin, and resveratrol. In addition, other methods of down regulating androgen receptor can include, but are not limited to, antisense compounds, ribozyme compounds, agents capable of methylation of CpG islands in the promoter regions that silence gene expression, agents capable of manipulating regulatory RNAs, and agents capable of gene therapy.

Where such forms exist, beta-diketones can include beta-diketone analogues, beta-diketone compound homologues, beta-diketone compound isomers, or beta-diketone derivatives thereof, that can function in a similar biological manner as beta-diketones to treat cancer and cancer-related conditions in a host. In addition, where such forms exist, beta-diketones can include pharmaceutically acceptable salts, esters, and prodrugs of the beta-diketones described or referred to above.

In particular beta-diketones can include, but are not limited to, dibenzoylmethane-type compounds, curcumin-type compounds, where such forms exist, and their respective analogues, homologues, isomers, and derivatives.

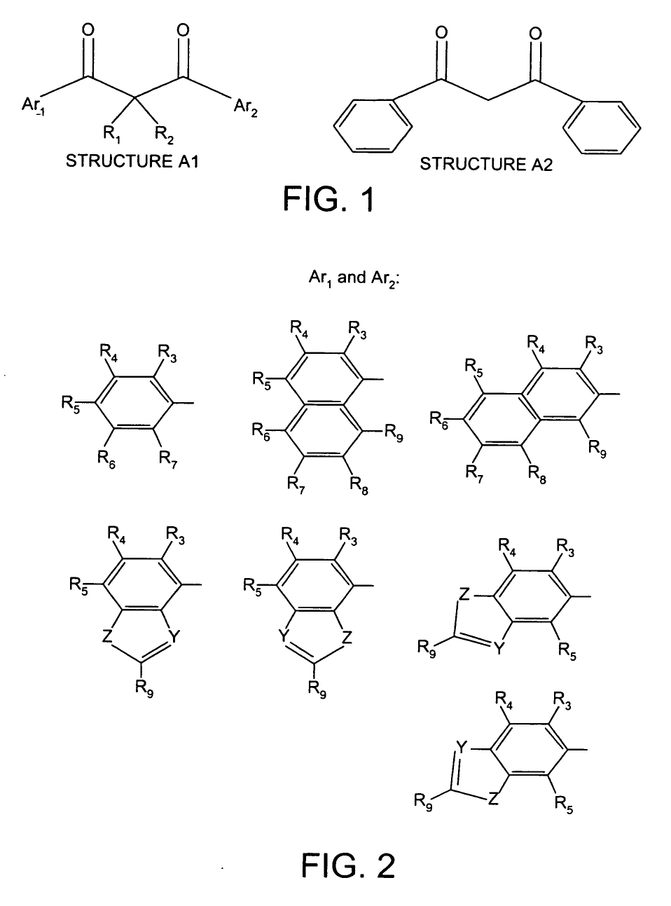

Dibenzoylmethane-type compounds can include, but are not limited to, structure A1 illustrated in FIG. 1. More particularly, dibenzoylmethane-type compounds can include dibenzoylmethane, which is illustrated as structure A2 in FIG. 1. Exemplary functional groups of the dibenzoylmethane-type compounds are indicated as Ar₁, Ar₂, R₁, and R₂. FIG. 2 illustrates exemplary functional groups (e.g., cyclic hydrocarbons and heterocyclic hydrocarbons) Ar₁ and Ar₂, which can include functional groups R₃, R₄, R₅, R₆, R₇, R₈, R₉, Y and Z. The functional groups R₁, R₂, R₃, R₄, R₅, R₆, R₇, R₈, and R₉ include, but are not limited to, hydrogen, alkyl groups, aryl groups, halo groups (F, Cl, Br, and I) hydroxy groups, alkoxy groups, alkylaminio groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons. The functional group Y includes, but is not limited to, nitrogen and CR₁, while Z includes, but is not limited to, oxygen, sulphur, NR₁, and CR₁.

Where such forms exist, dibenzoylmethane-type compounds can include, but are not limited to, dibenzoylmethane derivatives that function to treat cancer and cancer-related conditions in a host, and/or function prophylactically as a chemopreventative composition. In addition, where such forms exist, dibenzoylmethane-type compounds can include pharmaceutically acceptable salts, esters, and prodrugs of the dibenzoylmethane-type compounds described or referred to above.

Antisense compounds include, but are not limited to, compounds that may alter disease processes by blocking the production of harmful proteins by diseased cells via blocking RNA.

Ribozyme compounds include, but are not limited to, ribonucleic acid molecules with catalytic properties, used to damage nucleic acid sequences in diseased cells and preventing their gene products from being expressed.

By “pharmaceutically acceptable salt” it is meant those salts which are, within the scope of sound medical judgement, suitable for use in contact with the tissues of hosts without undue toxicity, irritation, allergic response and the like, and are commensurate with a reasonable benefit/risk ratio and effective for their intended use. The salts can be prepared in situ during the final isolation and purification of one or more effector agents, or separately by reacting the free base function with a suitable organic acid.

The term “pharmaceutically acceptable esters” as used herein refers to those esters of one or more effector agents which are suitable, within the scope of sound medical judgement, for use in contact with the tissues of hosts without undue toxicity, irritation, allergic response, and the like, are commensurate with a reasonable benefit/risk ratio, and are effective for their intended use.

The term “pharmaceutically acceptable prodrugs” as-used herein refers to those prodrugs of one or more effector agents which are, within the scope of sound medical judgement, suitable for use in contact with the tissues of hosts without undue toxicity, irritation, allergic response, and the like, are commensurate with a reasonable benefit/risk ratio, and are effective for their intended use. Pharmaceutically acceptable prodrugs also include zwitterionic forms, where possible, of one or more compounds of the composition. The term “prodrug” refers to compounds that are rapidly transformed in vivo to yield the parent compound, for example by hydrolysis in blood.

Effector agents and compositions (hereinafter “effector agents”) of this invention can be used to treat conditions such as, but not limited to, cancer and cancer-related diseases. In addition, effector agents of this invention can be used prophylactically as chemopreventative compositions that can be used to inhibit the development and/or slow the development of the cancer and cancer-related conditions and/or advanced stages of cancer and cancer-related conditions.

Examples of cancers and cancer-related conditions treatable by compositions of this invention include, but are not limited to, histologic types of cancer such as carcinoma, sarcoma, mesothelioma, and lymphoma including precancerous lesions. These cancers can develop at one or more bodily sites and these include, but are not limited to, head and neck, oral cavity and pharynx (e.g., tongue, mouth, and pharynx), cancer of the digestive system (e.g., esophagus, stomach, small intestine, colon, rectum, anus, anal canal, anorectum, liver and intrahepatic bile ducts, gallbladder and other sites in the biliary tree, pancreas and other digestive organs), respiratory system (e.g., larynx, lungs, bronchi, and other respiratory organs), bones and joints, soft tissues (e.g., heart), skin (e.g., basal cell, squamous cell and melaoma), breast, genital system (e.g., prostate, testis, penis, and other male genital organs as well as uterine cervix, uterine corpus, ovary, vulva, vagina and other female genital organs), urinary system (e.g., urinary bladder, kidney and renal pelvis, ureter and other urinary organs), brain and nervous system, eye and orbit, and endocrine system (e.g., thyroid and other endocrine). In addition, cancer can include non-site specific cancers such as, but not limited to, lymphoma (e.g., Hodgkin's disease and non-Hodgkin's lymphoma), leukemia (e.g., acute lymphocytic leukemia, chronic lymphocytic leukemia, acute myeloid leukemia, chronic myeloid leukemia, and other forms of leukemia), and multiple myeloma.

Effector agents of this invention can be used to treat these cancers and other cancers at any stage from the discovery of the cancer to advanced stages. In addition, effector agents of this invention can be used in the treatment of the primary cancer and metastases thereof.

Effector agents of this invention may be suitable for oral, rectal, nasal, topical (including buccal and sublingual), vaginal, or parenteral (including subcutaneous, intramuscular, subcutaneous, intravenous, intradermal, intraocular, intratracheal, intracistemal, intraperitoneal, and epidural) administration.

Effector agents may conveniently be presented in unit dosage form and may be prepared by conventional pharmaceutical techniques. Such techniques include the step of bringing into association one or more effector agents of the present invention and one or more pharmaceutical carriers or excipients.

Effector agents of the present invention suitable for oral administration may be presented as discrete units such as, but not limited to, tablets, caplets, pills or dragees capsules, or cachets, each containing a predetermined amount of one or more of the effector agents; as a powder or granules; as a solution or a suspension in an aqueous liquid or a non-aqueous liquid; or as an oil-in-water liquid emulsion or a water-in-oil emulsion or as a bolus, etc.

Effector agents of the present invention suitable for topical administration in the mouth include for example, lozenges, having the ingredients in a flavored basis, usually sucrose and acacia or tragacanth; pastilles, having one or more of the effector agents of the present invention in an inert basis such as gelatin and glycerin, or sucrose and acacia; and mouthwashes, having one or more of the effector agents of the present invention administered in a suitable liquid carrier.

Effector agents of the present invention suitable for topical administration to the skin may be presented as ointments, creams, gels, and pastes, having one or more of the effector agents administered in a pharmaceutically acceptable carrier.

Effector agents of the present invention for rectal administration may be presented as a suppository with a suitable base comprising, for example, cocoa butter or a salicylate.

Effector agents of the present invention suitable for nasal administration, when the carrier is a solid, include a coarse powder having a particle size, for example, in the range of 20 to 500 microns which is administered in the manner in which snuff is taken, (i.e., by rapid inhalation through the nasal passage from a container of the powder held close up to the nose). When the carrier is a liquid (for example, a nasal spray or as nasal drops), one or more of the effector agents can be admixed in an aqueous or oily solution, and inhaled or sprayed into the nasal passage.

Effector agents of the present invention suitable for vaginal administration may be presented as pessaries, tampons, creams, gels, pastes, foams or spray formulations containing one or more of the effector agents and appropriate carriers.

Effector agents of the present invention suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. Effector agents may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules, and tablets of the kind previously described above.

Pharmaceutical organic or inorganic solid or liquid carrier media suitable for enteral or parenteral administration can be used to fabricate the compositions. Gelatin, lactose, starch, magnesium stearate, talc, vegetable and animal fats and oils, gum, polyalkylene glycol, water, or other known carriers may all be suitable as carrier media.

Effector agents of the present invention may be used as the active ingredient in combination with one or more pharmaceutically acceptable carrier mediums and/or excipients. As used herein, “pharmaceutically acceptable carrier medium” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

Additionally, effector agents of the invention may be combined with pharmaceutically acceptable excipients, and, optionally, sustained-release matrices, such as biodegradable polymers, to form therapeutic compositions. A “pharmaceutically acceptable excipient” refers to a non-toxic solid, semi-solid or liquid filler, diluent, encapsulating material or formulation auxiliary of any type.

Except insofar as any conventional carrier medium is incompatible with the effector agents used in practicing embodiments of the invention, such as by producing any undesirable biological effect or otherwise interacting in a deleterious manner with one or more of the effector agents of the pharmaceutical composition, its use is contemplated to be within the scope of the embodiments of this invention.

When used in the above or other treatments, a therapeutically effective amount of one or more of the effector agents may be employed in pure form or, where such forms exist, in pharmaceutically acceptable salt, ester, and prodrug form. By a “therapeutically effective amount” of one or more of the effector agents it is meant a sufficient amount of one or more of the effector agents to treat cancer and cancer-related conditions at a reasonable benefit/risk ratio applicable to any medical treatment. For example, a “therapeutically effective amount” of one or more of the effector agents is an amount sufficient to palliate, ameliorate, stabilize, reverse, slow, and/or delay the progression or onset of the disease state compared to not administering one or more of the effector agents of the present invention.

It will be understood, however, that the total daily usage of the effector agents of the present invention will be decided by the attending physician within the scope of sound medical judgment. The specific therapeutically effective dose level for any particular host will depend upon a variety of factors, including for example, the disorder being treated and the severity of the disorder; activity of the specific effector agents employed; the specific effector agents employed, the age, body weight, general health, sex and diet of the patient; the time of administration; route of administration; rate of excretion of the specific effector agents employed; the duration of the treatment; drugs used in combination or coincidential with the specific composition employed; and like factors well known in the medical arts. For example, it is well within the skill of the art to start doses of the effector agents at levels lower than those required to achieve the desired therapeutic effect and to gradually increase the dosage until the desired effect is achieved.

Effector agents of the present inventions are preferably formulated in dosage unit form for ease of administration and uniformity of dosage. “Dosage unit form” as used herein refers to a physically discrete unit of the effector agents appropriate for the host to be treated. Each dosage should contain the quantity of effector agents calculated to produce the desired therapeutic affect either as such, or in association with the selected pharmaceutical carrier medium.

Preferred unit dosage formulations are those containing a daily dose or unit, daily sub-dose, or an appropriate fraction thereof, of the administered effector agent. In this regard, studies were performed to assess the dosage regimen for dibenzoylmethane (DBM).

LNCaP cells (commercially available from American Type Culture Collection of Rockville, Md., USA) are human prostate cancer cells that express a mutant, but functional, androgen receptor and exhibit increased proliferation in the response to androgens. LNCaP cells cultured in the presence of androgens secrete prostate specific antigen (PSA) into the culture medium. Intracellular and extracellular levels of PSA as well as androgen receptor levels were measured in LNCAP cells exposed to DBM. A dose-dependent down regulation of androgen receptor and PSA levels is observed in LNCaP cells exposed to DBM at 10, 25 and 50 micromolar concentrations. The androgen receptor levels are reduced to levels that are not readily detectable by Western analysis at DBM concentrations of 25 micromolar or higher. Intracellular PSA is reduced to levels that are not readily detectable by Western analysis at DBM concentrations of 10 micromolar or higher. Extracellular PSA assessed by ELISA analysis drops from about 700 nanograms per milliliter at baseline to approximately 200 nanograms per milliliter at micromolar DBM, 150 nanograms per milliliter at 25 micromolar DBM and 100 nanograms per milliliter at 50 micromolar DBM. These data suggest that DBM can down regulate the androgen receptor in human prostate cancer cells in a manner that is functionally relevant.

To further investigate the affect of DBM on prostate cancer, DBM was administered to TRAMP (TRansgenic Adenocarcinoma of Mouse Prostate) mice, TRAMP mice have been genetically altered to develop prostate cancer in a similar manner as the development and progression of human prostate cancer. In other words, development of premalignant prostatic intraepithelial (PIN) neoplasia and primary adenocarcinoma of the prostate of TRAMP mice is similar to the development of prostate cancer in humans. TRAMP mice are discussed in greater detail in U.S. Pat. No. 5,907,087, which is incorporated herein by reference.

Two groups of TRAMP mice were studied, where one is a control cohort and the other is the cohort fed DBM. In general, TRAMP mice fed ab lib a diet of 1% DBM (weight/weight) mixed with mice food (AIN-76A) from six weeks of age are less likely to develop either adenocarcinoma or high grade PIN (2 of 11) than mice in the control cohort (6 of 10). In particular, TRAMP mice fed a diet of 1% DBM developed adenocarcinoma. In addition, TRAMP mice fed a diet of 1% DBM from six weeks of age achieve serum levels of 1 micromolar DBM with a range of 0.49 to 1.91 micromolar DBM for four mice.

These results indicate that oral DBM is absorbed sufficiently from the gastrointestinal tract (i.e., orally bioavailable) of TRAMP mice to yield serum levels that are of a similar order of magnitude as those we have shown to inhibit proliferation of human prostate cancer cells in vitro, as discussed above. In addition, TRAMP mice exposed to 1% DBM in the manner described have significantly less cancer and high-grade prostatic intraepithelial neoplasia at 18 weeks than mice in the control cohort. Therefore, adequate amounts of DBM entered the prostate in TRAMP mice to inhibit carcinogenesis.

In combination, these two studies indicate that down regulation of the androgen receptor should lead to a corresponding down regulation of androgen-responsive genes, which is correlated to prostate cancer. Likewise, the TRAMP mice study indicates that DBM is absorbed by the gastrointestinal tract of the TRAMP mice and sufficient amounts of DBM entered the prostate to inhibit carcinogenisis. Thus, a dosage regime for DBM to treat humans for prostate cancer can be developed.

Based on the foregoing studies, a dosage regime for DBM and other effector agents can be developed. In general, the starting dose of most Phase I clinical trials is based on preclinical testing, and is usually quite conservative. A standard measure of toxicity of a drug in preclinical testing is the percentage of animals (rodents) that die because of treatment. The dose at which 10% of the animals die is known as the LD₁₀, which has in the past often correlated with the maximal-tolerated dose (MTD) in humans, adjusted for body surface area. The adjustment for body surface area includes host factors such as, for example, surface area, weight, metabolism, tissue distribution, absorption rate, and excretion rate. Thus, the standard conservative starting dose is one tenth the murine LD₁₀, although it may be even lower if other species (i.e., dogs) were more sensitive to the drug. It is anticipated that a starting dose for DBM and other effector agents in Phase I clinical trials in humans will be determined in this manner. This dosing regimen is discussed in more detail in Freireich E J, et al., Cancer Chemother Rep 50:219-244, 1966, which is incorporated herein by reference.

As stated above, a therapeutically effective dose level will depend on many factors. In addition, it is well within the skill of the art to start doses of the effector agents at relatively low levels, and increase the dosage until the desired effect is achieved.

Effector agents of the present invention may be used in combination with other effector agents and/or procedures for the treatment of the conditions described above. For example, a tumor may be treated conventionally with surgery, radiation, or chemotherapy combined with one or more effector agents of the present invention and then one or more effector agents of the present invention may be subsequently administered to the patient to extend the dormancy of micrometastases and to stabilize, inhibit, or reduce the growth of any residual primary tumor.

In particular, effector agents of the invention may also be combined with other anticancer agents to enhance their effectiveness, or combined with other anticancer agents and administered together with other cytotoxic and cytostatic agents. In particular, when used to treat cancer, effector agents of the invention may be administered with angiostatin, BCNU, biclutamide, bisphonates, bleomycin, busulfan, carboplatin, 2-chlorodeoxyadenosine, cisplatin, cyclophosphomide, cytosine arabinoside, dacarbazine, daunorubicin, dexamethasone, docetaxel, doxorubicin, endostatin, epirubicin, estramustine, etoposide, fludarabine, 5-fluorouracil, flutamide, gemcitabine, interferons, herceptin, hydroxyurea, ifosfamide, interleukin-2, interleukin-12, irinotecan, leuprolide, melphalan, 6-mercaptopurine, methotrexate, mitoxantrone, nitrogen mustard, oxaliplatin, nilutamide, paclitaxel, prednisone, procarbazine, retinoids, rituxan and other therapeutics antibodies, tamoxifen, thalidomide, thiotepa, topotecan, vinblastine, vincristine, as well as with radiation.

Effector agents of the present invention may be used with a sustained-release matrix. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

As indicated above, effector agents of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The liposome can contain, in addition to one or more compositions of the present invention, stabilizers, preservatives, excipients, and the like. The preferred lipids are the phospholipids and the phosphatidyl cholines (lecithins), both natural and synthetic. Methods to form liposomes are known in the art.

It should be emphasized that the above-described embodiments of the present invention are merely possible examples of implementations, and are set forth only for a clear understanding of the principles of the invention. Many variations and modifications may be made to the above-described embodiments of the invention without departing substantially from the spirit and principles of the invention. All such modifications and variations are intended to be included herein within the scope of this disclosure and the present invention and protected by the following claims.

Therefore, having thus described the invention, at least the following is claimed:

1. A method of treating cancer comprising administering to a host in need of treatment an effective amount of at least one dibenzoylmethane-type compound.

2. The method of claim 1, wherein the at least one dibenzoylmethane-type compound includes dibenzoylmethane.

3. The method of claim 1, wherein the cancer is chosen from breast cancer and genital cancer.

4. The method of claim 1, wherein the cancer is prostate cancer.

5. The method of claim 1, further comprising treating the host with at least one conventional anticancer treatment chosen from radiation and chemotherapy.

6. The method of claim 1, further comprising treating the host with at least one conventional anticancer agent.

7. The method of claim 1, wherein at least one dibenzoylmethane-type compound includes pharmaceutically acceptable salts of the dibenzoylmethane-type compounds.

8. The method of claim 1, wherein the at least one dibenzoylmethane-type compound includes pharmaceutically acceptable prodrugs of the dibenzoylmethane-type compounds.

9. The method of claim 1, wherein the administration of at least one dibenzoylmethane-type compound is orally bioavailable.

10. The method of claim 1, wherein the at least one dibenzoylmethane-type compound has the formula of structure A1: 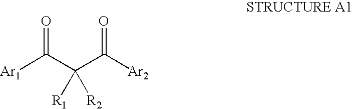
wherein Ar₁ and Ar₂ include 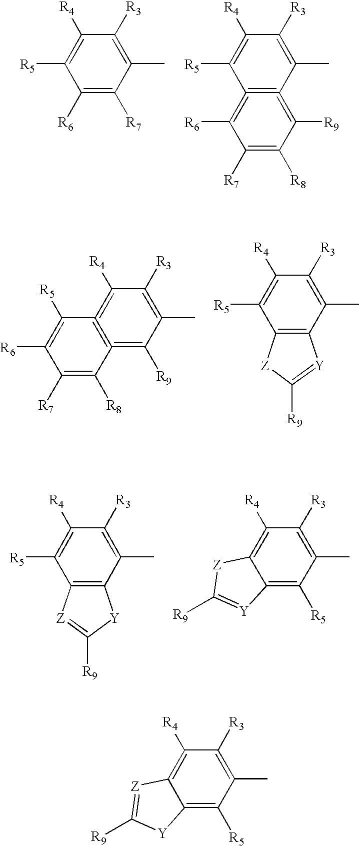
wherein R₁, R₂, R₃, R₄, R₅, R₆, R₇, R₈, and R₉ include hydrogen, alkyl groups, aryl groups, halo groups, hydroxy groups, alkoxy groups, alkylaminio groups, dialkylamino groups, acyl groups, carboxyl groups, carboamido groups, sulfonamide groups, aminoacyl groups, amide groups, amine groups, nitro groups, organo selenium compounds, hydrocarbons, and cyclic hydrocarbons, wherein Y includes nitrogen and CR₁, and wherein Z includes oxygen, sulfer, NR₁, and CR₁.

11. A chemopreventative method of prophylactically treating cancer comprising administering to a host in need of treatment an effective amount of at least one dibenzoylmethane-type compound.

12. The chemopreventative method of claim 11, wherein the at least one dibenzoylmethane-type includes dibenzoylmethane.

13. The chemopreventative method of claim 11, wherein the cancer is chosen from breast cancer and genital cancer.

14. A pharmaceutical composition comprising at least one dibenzoylmethane-type compound in combination with a pharmaceutically acceptable carrier, wherein the at least one dibenzoylmethane-type compound is present in a dosage level effective to treat cancer.

15. The pharmaceutical composition of claim 14, wherein the at least one dibenzoylmethane-type compound is orally bioavailable.

16. The pharmaceutical composition of claim 14, wherein the at least one dibenzoylmethane-type compound includes pharmaceutically acceptable salts of the dibenzoylmethane-type compound.

17. The pharmaceutical composition of claim 14, wherein the at least one dibenzoylmethane-type compound includes pharmaceutically acceptable prodrugs of the dibenzoylmethane-type compound.

18. The pharmaceutical composition of claim 14, wherein pharmaceutical composition can be administered orally, rectally, parenterally, intrasystemically, intravaginally, intraperitoneally, topically, and bucally.

19. A method of decreasing androgen-associated cancer by down regulating androgen receptors present in host cells, comprising exposing the host cells to an effective amount of at least one effector agent.

20. The method of claim 19, wherein the effector agent includes at least one dibenzoylmethane-type compound.

21. The method of claim 19, wherein the effector agent is chosen from antisense compounds and ribozyme compounds.

22. The method of claim 19, wherein the androgen-associated cancer is chosen from breast cancer and genital cancer.

23. The method of claim 19, wherein the effector agent includes dibenzoylmethane.

24. The method of claim 19, wherein the host cells are cancerous cells.

25. The method of claim 19, wherein the host cells are non-cancerous cells.

26. A method of down regulating androgen receptors, comprising administering an effective amount of at least one effector agent to a host in need of treatment.

27. The method of claim 26, wherein the effector agent includes at least one dibenzoylmethane-type compound.

28. The method of claim 26, wherein the effector agent includes dibenzoylmethane.

29. A method of down regulating prostate-specific antigen (PSA) levels in human prostate cancer cells, comprising administering an effective amount of at least one effector agent to a host in need of treatment.

30. The method of claim 29, wherein the effector agent includes at least one dibenzoylmethane-type compound.

31. The method of claim 30, wherein the effector agent includes dibenzoylmethane.

32. A pharmaceutical composition comprising at least one effector agent in combination with a pharmaceutically acceptable carrier, wherein the at least one effector agent is present in a dosage level effective to down regulate androgen receptors present in host cells.

33. The method of claim 32, wherein the host cells are cancerous cells.

34. The method of claim 32, wherein the host cells are non-cancerous cells.

35. The method of claim 32, wherein the effector agent includes at least one dibenzoylmethane-type compound.

36. The method of claim 32, wherein the effector agent includes dibenzoylmethane.

37. The method of claim 32, wherein the effector agent includes at least one dibenzoylmethane-type compound.
